SEQUENCE LISTING (46 sequences)

SEQ ID NO: 1 - protein, 488 aa, Schizosaccharomyces pombe
MSNTNFSTSRSSKSIPELPNLEALRSLWPPPSLNESGDTRSVWTTHTGEPVASSVLSTSGSNNFSSPLKRPAPESHDAPIGRRLMVDDPRLIKHGKYDFSRHCTDYGHSYEWPYFRSLRRESMLYHTSGSYPESQPPYSSYSTDAPHYYHAGSESSAYYDSRSRLHGIQPPPKRRTLSPPPRRLADPVVVGSSRYVEEEVYRRPPYTLASEVPSSASAYQAGYSSYPVRSSPQLSHEDTRHGIASSGSTRYPFVPANTRASHSPSLLEPYAHSLPSSVAPVGAYPEKSSYLLSNSSNDSASRKEKPKARASTPPPLNFSRASEHRNEKGERISMINPRVVLDENGISHRSRYFIMLCDNETAIAHAKKTSIWAVKKDSSKRISDAYKKASVYFIFVAQQTYNALGYAQVVSDLNSTELPFWSDSSHAGGVRIKWIKTCNLFSAEISEIVSHMDHGSEARDGMEMMYDEGSRLCTLINYAIMKRIGRDR

SEQ ID NO: 2 - DNA, 1467 bp, Schizosaccharomyces pombe
  source 1..1467: mol_type = genomic DNA
atgtcaaacacaaacttctctacttctaggtcatcaaagtccattccggaacttccaaatttggaagctctgcgaagcctgtggcctccaccgtcattgaacgaatcgggcgatactagaagcgtatggactactcataccggagagcctgttgcgtcaagtgttctatctacttcaggttcaaacaacttttcttcacctctgaaaagacctgcaccagagtcacatgacgctcctatcggtagaagattgatggtagatgatccccgcttaattaaacatggtaaatatgattttagcaggcattgcactgactatggtcactcctacgaatggccttattttcgatcccttcgtcgtgagtctatgctttatcacacttcgggatcttatccagaaagtcagcctccttactcttcttattcaactgatgctccacattattatcatgcaggttcggaatcctctgcttactatgatagccgttctcgtttgcatggcatacaacctcctcccaagcgacgcacacttagtcctccccctagacggcttgcggacccagtggttgttggaagttctcgttatgttgaagaagaagtatacagaaggcctccttacactttagcttctgaagttccttcatccgcttcagcataccaagctggttactcttcctatcctgttcgatcttccccccaactttctcatgaagacactcgtcatggtattgcaagcagtggttcaacaaggtatccttttgtacctgctaatactcgtgcatctcactcccctagtttgcttgaaccttatgctcactcccttccctcttcggtcgcgccagttggagcttatcctgagaaatcttcttacctgctttctaattccagcaatgatagtgcctcaagaaaagaaaaacccaaagctcgtgcatctacgcctcctccattaaatttttccagagcaagcgaacatagaaatgagaagggggaacgcatttcgatgattaatccccgagttgttcttgacgaaaatggaatttcacaccgctcccgttattttattatgctatgtgacaacgagactgccattgctcatgctaaaaaaacttctatttgggcagtcaagaaagattcttccaaaagaatttctgatgcatataagaaagccagtgtttattttatttttgtggctcagcaaacgtacaatgctttaggatatgctcaggtagtcagcgacctaaatagcaccgaactgcccttttggtcagatagctcgcatgctggaggtgttcgcatcaagtggattaaaacttgcaatctgttttctgctgaaatttctgaaattgttagccacatggatcatgggtctgaagctagagatggcatggaaatgatgtatgatgaaggcagcagactttgtacgttgattaactatgctataatgaagcgaattggaagagaccgttga

SEQ ID NO: 3 - protein, 479 aa, Schizosaccharomyces japonicus
MSNSNASLQAAAIPSMEALRTLWRNIAPSTSGDSQEARSVWAKHPNDPTVPAAAPAAAGLFSSPLKRGAPDSKEYMDRHVLSEPVSVSSATVYDFYRHCTDYGHSYDWPYFRSLRRELAYRNSVDAAPYPPYRPEPSPRYAVPPYGQRSPTNYPMRYEESLQSAGQPPLKRRTLLSPHLSGMNSRLMMPSRYEDEYYTRPYNYGKAPAQASSFQSTMHSNGYTPARPESVPESAGIHGYHPMRQDAGDSPLFSEPTAHTEGYRDFDPLHGKFAPPSSHSYGIEPYDHKAEPRREKPKTRAPTPPPLDFEHAHEYRNEKGERVSMINPRVILDENGISHRSRYFIMLVDNETAISHAKRSSVWSLKQNYANAISEAYKQEANVYFIFINAKSYNALGYAQLTSDVGTVAKPFWADTTYTNGVNVKWIKTCNLYSNEISEIVSRMDHGARARDGQEMMYDEGSRLCTLINSAIMKRIGRDR

SEQ ID NO: 4 - DNA, 1440 bp, Schizosaccharomyces japonicus
  source 1..1440: mol_type = genomic DNA
atgtcgaattccaatgcttcactgcaagcggctgccataccgagtatggaggcgttaagaacgctatggcgtaacatagctccttccacttccggcgattcacaagaggcccggagtgtttgggctaagcatccaaatgatcctacggttcctgctgctgctccagctgccgcaggtcttttttcctctccattgaaacgcggtgctcctgattcaaaagagtatatggatcgtcatgtgctttcagagcctgtttctgtgagcagtgctactgtgtatgacttctaccgtcactgcactgattacggccattcttatgattggccctacttccgttcattgcgtcgcgagttggcataccgcaattctgtcgacgctgctccttatcctccgtatcgcccagaacccagtccccgctacgcagtacctccgtatggtcaacgttctccgacaaattatcccatgcgttatgaagagtctttgcagtctgcaggacagcccccgctaaaacgtcgtactttattaagtccacatttaagtggcatgaactctcggttaatgatgccttcacggtatgaagatgagtattacacgagaccttataattatggtaaggctcctgcccaagcgtcctcttttcaaagtaccatgcattccaatggctacactcctgcacgaccagaaagtgtcccggaatcagctggaatacatggataccaccctatgcggcaggatgctggcgattcaccattgttttcggaaccaactgctcatactgaagggtatcgcgatttcgatcctttacacggaaagtttgctcccccgtcctctcattcgtatggcatcgagccttatgaccataaagccgaacctcgtcgtgaaaaacccaaaacacgagcacctacaccccctccgttggattttgagcatgctcacgaatatcgcaatgaaaagggtgaacgtgtctccatgatcaatccacgtgttatccttgatgaaaatggaatctcacataggtcgcggtacttcatcatgctggtggataatgaaacagcaattagtcatgccaaacgaagttctgtttggagtcttaaacaaaattatgcgaacgctatcagcgaagcttacaagcaagaagcaaatgtctattttattttcatcaatgccaagtcctataatgcactggggtatgctcagcttacttctgatgtcgggactgttgcgaaaccgttttgggcagataccacatacactaatggtgtaaatgtcaagtggattaaaacgtgtaacttgtattcaaacgaaatttcagaaattgtgagccgcatggatcacggtgctcgggctagagatggtcaagaaatgatgtacgacgaaggaagtcgtctctgtaccttgatcaattcggcgattatgaaacgtattggtagagatcgttga

SEQ ID NO: 5 - protein, 480 aa, Schizosaccharomyces octosporus
MSNTNFNPQSSKQLPELPNLEVLRSLWPSQPSNEPNDSRSVWSNRPAEPVHVLSNPAANSFTSPLKRPAPDSREAPMGRRIMVDDPRLAKAVKYDFTRHCTDYGHSYEWPYFRSVRRESLMYGPPPAAVPPESPVFPPYSAEPASLQPKYYPPESDIPATFYDRARFGLQPPSKRRTLSPPPRRVVDPYGPHFVEEDPYSKSSYALAGDRPPLSAPMYPVGYPPYSAKAPSQIAADDFRGTASRYPLTASDVRASHSPGLIDPYTHRPVADVAYADRSHYAMNAEPYSAPPPRKEKPKPRAPTPPPLNFARASEYRNDKGERISMINPRVILDENGISSRSRYFIMLCDNETAISHAKKTSIWAVKHDAASRVSDAYKNASIYFIFIAKPTNNALGYAQVVSDLNSAELPFWADNATYAGGVRVKWIKTCNLFSAEISDIVGRMNHGATARDGMEMMYDEGCRLCILVNSAIMKRIGRDR

SEQ ID NO: 6 - DNA, 1443 bp, Schizosaccharomyces octosporus
  source 1..1443: mol_type = genomic DNA
atgtcaaacacaaactttaatcctcagtcttctaaacaattacctgaactcccaaacttggaggtattacgaagcttgtggccttctcaaccatcaaacgaacctaatgactccaggagtgtctggtctaacagaccggcggagcctgtgcatgtgctatcaaatcctgctgcgaatagttttacttctcctttaaaacgtccagctcctgattctcgtgaagctcctatgggaagaagaattatggttgatgatccacgcttagcgaaagctgtgaagtacgattttacccggcactgtacggattatggtcattcttatgaatggccttattttcgttctgttcgtcgtgaatcgttgatgtatggtccaccacctgctgctgttcctcctgaatctcctgtttttccaccctattctgctgaaccagctagcctacaacccaaatactatccacccgagtctgatattcccgccacattttatgatcgtgctcgttttggactgcagcccccttcaaaacgtagaacattgagtcctcctccaagaagagttgttgatccatatggccctcactttgttgaggaggatccatactcaaaatcctcttacgcattggctggggatcgtcctcctttatcagcccccatgtatcctgttggctatcctccgtatagtgcgaaagctccttcccaaatagctgctgatgattttcgtgggactgctagtagatatcctttgacggctagtgacgttcgggcttctcattctcctggactcattgatccatatactcatcgtcctgttgcagacgttgcttatgcggatcgttctcattatgctatgaatgcagagccttattcggctcctcctcctagaaaggaaaaaccaaagccacgtgctcctacacctcctccattaaattttgctagagccagtgagtacagaaatgacaaaggtgaacggatatctatgatcaatcctcgtgtaattcttgatgaaaatggaatatctagccgctctcgttattttataatgctttgtgataacgagacggctatttctcatgcaaagaaaacgtccatttgggctgtaaagcacgatgcagcatcccgtgtatcagatgcttacaagaatgctagtatttatttcatttttattgctaagccaacaaacaacgctcttggctatgctcaagttgttagtgatttgaatagcgcggagcttccgttttgggcagataatgctacctatgctggaggcgttcgcgtgaagtggattaaaacttgcaatcttttctctgccgagatatctgatatcgttggacgaatgaaccatggagcaaccgctagagatggaatggaaatgatgtacgatgaaggttgccgtctttgcatattggttaactcggccatcatgaagcgaatcggtcgtgatcggtaa

SEQ ID NO: 7 - protein, 479 aa, Schizosaccharomyces cryophilus
MSNTNFNSQSSKQLPELPNLEVLRSLWPSQPSNESNDSRSVWSSRPAEPVHVLSNPTANSFTSPLKRPAPDSREAPIGRRIMIDDPRLAKPVKYDFTRHCTDYGHSYEWPYFRSVRRESLMYGPPSAVPPESPVFPPYPPESATLQSKYYPAQSDAPAAYYDRARFELQPPSKRRTLSPPPRRIVDPYGPRFTEDDPYSKSPYAMAGDRPPLSAPMYPVGYPPYGAKASSQLGADDLRGTTSRYPLMASDVRASHSPSLIDPYAHRSVADVPYADHPHYSVNAEPYSAPPPRKEKPKPRAPTPPPLNFARASEYRNDKGERISMINPRVILDESGISHRSRYFIMLCDNETAIAHAKKTSIWAVKHEAASRVSDAYKKASIYFIFIAKPTNNALGYAQVVSDLNSAELPFWADNATYAGGVRVKWIKTCNLFSAEISDIVGRMNHGMTAKDGMEMMYDEGCRLCILVNSAIMKRIGRDR

SEQ ID NO: 8 - DNA, 1440 bp, Schizosaccharomyces cryophilus
  source 1..1440: mol_type = genomic DNA
atgtcaaacacaaactttaattctcagtcttctaaacaattacctgaactcccaaacttggaggttttgcgaagcttatggccttctcaaccgtcaaacgaatctaatgactctagaagtgtctggtcaagcagaccagcggaacccgtccatgtactttcaaatcctactgcgaatagttttacttctcctttaaaaagaccggctcccgattctcgtgaggcccccattggaagaagaattatgattgatgatccacgcttagcgaagccagtgaaatatgattttactcgccattgtactgattatggccattcttatgagtggccttactttcgctctgttcgtcgtgaatcgttaatgtatggaccgccttctgctgttcctcctgagtctcctgttttcccaccatatcctcccgagtcagctacgctgcaatccaaatactatccagcccaatctgatgcacctgccgcgtattatgatcgcgctcgttttgaattgcaacctccttcaaagcgaagaacattgagtcctccaccaaggagaatcgttgacccatatggtcctcgttttactgaggatgatccctactcaaaatcaccatatgcaatggctggcgatcgtcctccattatcagctcccatgtatcctgttggctatcctccgtatggtgcgaaagcttcctctcagcttggtgctgatgatttgcgtggtaccactagtagatatcccttgatggctagtgatgttcgtgcttctcattctcctagccttattgacccttatgcccatcgttctgttgcagatgttccttatgcggatcaccctcattactcagtgaacgctgagccttattcagcacctcctcccagaaaggaaaaacctaagccgcgtgctcccactccccctccgctaaattttgcgagagcaagtgaatatagaaatgacaaaggcgaacggatatctatgattaaccctcgtgtgattcttgacgaaagtggaatttcacatcgctctcgttattttattatgctttgtgacaacgaaacagcgattgcccatgcaaagaaaacctctatttgggctgtaaagcacgaagcagcttctcgtgtatctgatgcatataagaaggctagtatctattttatttttattgcaaagccgacaaataatgctcttggttatgctcaagtcgtgagtgatttgaacagtgcagaactccctttttgggcagataatgctacatacgccggaggcgttcgtgtgaaatggattaaaacttgcaatcttttctctgctgaaatctctgatatcgttggacgaatgaatcacggaatgactgctaaagatggaatggaaatgatgtacgacgagggatgccgcctttgtattttggttaactcggccatcatgaagcgaattggacgtgatcggtaa

SEQ ID NO: 9 - protein, 173 aa, Schizosaccharomyces pombe
LNFSRASEHRNEKGERISMINPRVVLDENGISHRSRYFIMLCDNETAIAHAKKTSIWAVKKDSSKRISDAYKKASVYFIFVAQQTYNALGYAQVVSDLNSTELPFWSDSSHAGGVRIKWIKTCNLFSAEISEIVSHMDHGSEARDGMEMMYDEGSRLCTLINYAIMKRIGRDR

SEQ ID NO: 10 - protein, 174 aa, Schizosaccharomyces japonicus
LDFEHAHEYRNEKGERVSMINPRVILDENGISHRSRYFIMLVDNETAISHAKRSSVWSLKQNYANAISEAYKQEANVYFIFINAKSYNALGYAQLTSDVGTVAKPFWADTTYTNGVNVKWIKTCNLYSNEISEIVSRMDHGARARDGQEMMYDEGSRLCTLINSAIMKRIGRDR

SEQ ID NO: 11 - protein, 174 aa, Schizosaccharomyces octosporus
LNFARASEYRNDKGERISMINPRVILDENGISSRSRYFIMLCDNETAISHAKKTSIWAVKHDAASRVSDAYKNASIYFIFIAKPTNNALGYAQVVSDLNSAELPFWADNATYAGGVRVKWIKTCNLFSAEISDIVGRMNHGATARDGMEMMYDEGCRLCILVNSAIMKRIGRDR

SEQ ID NO: 12 - protein, 174 aa, Schizosaccharomyces cryophilus
LNFARASEYRNDKGERISMINPRVILDESGISHRSRYFIMLCDNETAIAHAKKTSIWAVKHEAASRVSDAYKKASIYFIFIAKPTNNALGYAQVVSDLNSAELPFWADNATYAGGVRVKWIKTCNLFSAEISDIVGRMNHGMTAKDGMEMMYDEGCRLCILVNSAIMKRIGRDR

SEQ ID NO: 13 - protein, 11 aa, synthetic construct
  REGION 1..11: note = Domain 2 of Mmi1 of Schizosaccharomyces
  VARIANT 5: note = T, S, or A
  VARIANT 6: note = T, R, K, S
  VARIANT 7: note = H or R
  VARIANT 8: note = T or P
  VARIANT 9: note = G, A, or R
  VARIANT 10: note = E or D
RSVWXXXXXXP

SEQ ID NO: 14 - protein, 19 aa, synthetic construct
  REGION 1..19: note = Domain 3 of Mmi1 of Schizosaccharomyces
  VARIANT 2: note = S or T
  VARIANT 8: note = P or G
  VARIANT 11: note = E or D
  VARIANT 13: note = H, R, or K
  VARIANT 14: note = D or E
  VARIANT 15: note = A or Y
  VARIANT 16: note = null or P
  VARIANT 17: note = I or M
  VARIANT 18: note = G or D
FXSPLKRXAPXSXXXXXXR

SEQ ID NO: 15 - protein, 29 aa, synthetic construct
  REGION 1..29: note = Domain 4 of Mmi1 of Schizosaccharomyces
  VARIANT 4: note = S, T, or Y
  VARIANT 15: note = E or D
  VARIANT 22: note = L or V
  VARIANT 26: note = null or S
  VARIANT 27: note = L or M
  VARIANT 28: note = R, L, or M
YDFXRHCTDYGHSYXWPYFRSXRREXXXY

SEQ ID NO: 16 - protein, 12 aa, synthetic construct
  REGION 1..12: note = Domain 5 of Mmi1 of Schizosaccharomyces
  VARIANT 4: note = P, S, or L
  VARIANT 10: note = S or L
  VARIANT 11: note = P or S
QPPXKRRTLXXP

SEQ ID NO: 17 - protein, 14 aa, synthetic construct
  REGION 1..14: note = Domain 6 of Mmi1 of Schizosaccharomyces
  VARIANT 1: note = R or D
  VARIANT 3: note = S or G
  VARIANT 4: note = H or D
  VARIANT 7: note = S, G, or L
  VARIANT 8: note = L or F
  VARIANT 9: note = L, I, or S
  VARIANT 10: note = E or D
  VARIANT 12: note = Y or T
  VARIANT 13: note = A or T
XAXXSPXXXXPXXH

SEQ ID NO: 18 - protein, 14 aa, synthetic construct
  REGION 1..14: note = Domain 7 of Mmi1 of Schizosaccharomyces
  VARIANT 2: note = K or R
  VARIANT 7: note = A, P, or T
  VARIANT 10: note = S or P
RXEKPKXRAXTPPP

SEQ ID NO: 19 - protein, 11 aa, Schizosaccharomyces pombe
RSVWTTHTGEP

SEQ ID NO: 20 - protein, 19 aa, Schizosaccharomyces pombe
FSSPLKRPAPESHDAPIGR

SEQ ID NO: 21 - protein, 29 aa, Schizosaccharomyces pombe
YDFSRHCTDYGHSYEWPYFRSLRRESMLY

SEQ ID NO: 22 - protein, 12 aa, Schizosaccharomyces pombe
QPPPKRRTLSPP

SEQ ID NO: 23 - protein, 14 aa, Schizosaccharomyces pombe
RASHSPSLLEPYAH

SEQ ID NO: 24 - protein, 14 aa, Schizosaccharomyces pombe
RKEKPKARASTPPP

SEQ ID NO: 25 - protein, 458 aa, Schizosaccharomyces pombe
PSLNESGDTRSVWTTHTGEPVASSVLSTSGSNNFSSPLKRPAPESHDAPIGRRLMVDDPRLIKHGKYDFSRHCTDYGHSYEWPYFRSLRRESMLYHTSGSYPESQPPYSSYSTDAPHYYHAGSESSAYYDSRSRLHGIQPPPKRRTLSPPPRRLADPVVVGSSRYVEEEVYRRPPYTLASEVPSSASAYQAGYSSYPVRSSPQLSHEDTRHGIASSGSTRYPFVPANTRASHSPSLLEPYAHSLPSSVAPVGAYPEKSSYLLSNSSNDSASRKEKPKARASTPPPLNFSRASEHRNEKGERISMINPRVVLDENGISHRSRYFIMLCDNETAIAHAKKTSIWAVKKDSSKRISDAYKKASVYFIFVAQQTYNALGYAQVVSDLNSTELPFWSDSSHAGGVRIKWIKTCNLFSAEISEIVSHMDHGSEARDGMEMMYDEGSRLCTLINYAIMKRIGRDR

SEQ ID NO: 26 - protein, 11 aa, Schizosaccharomyces japonicus
RSVWAKHPNDP

SEQ ID NO: 27 - protein, 18 aa, Schizosaccharomyces japonicus
FSSPLKRGAPDSKEYMDR

SEQ ID NO: 28 - protein, 28 aa, Schizosaccharomyces japonicus
YDFYRHCTDYGHSYDWPYFRSLRRELAY

SEQ ID NO: 29 - protein, 12 aa, Schizosaccharomyces japonicus
QPPLKRRTLLSP

SEQ ID NO: 30 - protein, 14 aa, Schizosaccharomyces japonicus
DAGDSPLFSEPTAH

SEQ ID NO: 31 - protein, 14 aa, Schizosaccharomyces japonicus
RREKPKTRAPTPPP

SEQ ID NO: 32 - protein, 11 aa, Schizosaccharomyces octosporus
RSVWSNRPAEP

SEQ ID NO: 33 - protein, 19 aa, Schizosaccharomyces octosporus
FTSPLKRPAPDSREAPMGR

SEQ ID NO: 34 - protein, 29 aa, Schizosaccharomyces octosporus
YDFTRHCTDYGHSYEWPYFRSVRRESLMY

SEQ ID NO: 35 - protein, 12 aa, Schizosaccharomyces octosporus
QPPSKRRTLSPP

SEQ ID NO: 36 - protein, 14 aa, Schizosaccharomyces octosporus
RASHSPGLIDPYTH

SEQ ID NO: 37 - protein, 14 aa, Schizosaccharomyces octosporus
RKEKPKPRAPTPPP

SEQ ID NO: 38 - protein, 11 aa, Schizosaccharomyces cryophilus
RSVWSSRPAEP

SEQ ID NO: 39 - protein, 19 aa, Schizosaccharomyces cryophilus
FTSPLKRPAPDSREAPIGR

SEQ ID NO: 40 - protein, 29 aa, Schizosaccharomyces cryophilus
YDFTRHCTDYGHSYEWPYFRSVRRESLMY

SEQ ID NO: 41 - protein, 12 aa, Schizosaccharomyces cryophilus
QPPSKRRTLSPP

SEQ ID NO: 42 - protein, 14 aa, Schizosaccharomyces cryophilus
RASHSPSLIDPYAH

SEQ ID NO: 43 - protein, 14 aa, Schizosaccharomyces cryophilus
RKEKPKPRAPTPPP

SEQ ID NO: 44 - DNA, 519 bp, Schizosaccharomyces pombe
  source 1..519: mol_type = genomic DNA
aatttttccagagcaagcgaacatagaaatgagaagggggaacgcatttcgatgattaatccccgagttgttcttgacgaaaatggaatttcacaccgctcccgttattttattatgctatgtgacaacgagactgccattgctcatgctaaaaaaacttctatttgggcagtcaagaaagattcttccaaaagaatttctgatgcatataagaaagccagtgtttattttatttttgtggctcagcaaacgtacaatgctttaggatatgctcaggtagtcagcgacctaaatagcaccgaactgcccttttggtcagatagctcgcatgctggaggtgttcgcatcaagtggattaaaacttgcaatctgttttctgctgaaatttctgaaattgttagccacatggatcatgggtctgaagctagagatggcatggaaatgatgtatgatgaaggcagcagactttgtacgttgattaactatgctataatgaagcgaattggaagagaccgttga

SEQ ID NO: 45 - RNA, 18 nt, synthetic construct
  misc_feature 1..18: note = Substrate for Mmi1 binding
  misc_feature 1: note = Biotinylation
  source 1..18: mol_type = other RNA
ggatccttaaacagatct

SEQ ID NO: 46 - RNA, 18 nt, synthetic construct
  misc_feature 1..18: note = Negative control for Mmi1 binding
  misc_feature 1: note = Biotinylation
  source 1..18: mol_type = other RNA
ggatccctaaacagatct

(19) United States
(12) Patent Application Publication
(10) Pub. No.: US 2025/0034205 A1
(43) Pub. Date: Jan. 30, 2025

(54) METHOD FOR PURIFYING A PROTEIN OF INTEREST AND MEANS FOR ITS IMPLEMENTATION

(71) Applicants: UNIVERSITÉ GRENOBLE ALPES, SAINT MARTIN D'HÈRES (FR); CENTRE NATIONAL DE LA RECHERCHE SCIENTIFIQUE, Paris (FR); INSTITUT NATIONAL DE LA SANTÉ ET DE LA RECHERCHE MÉDICALE, Paris (FR)

(72) Inventors: André VERDEL, Biviers (FR); Leïla TODESCHINI, Le Sappey-en-Chartreuse (FR); Daniel PERAZZA, Sinard (FR)

(73) Assignees: UNIVERSITÉ GRENOBLE ALPES, SAINT MARTIN D'HÈRES (FR); CENTRE NATIONAL DE LA RECHERCHE SCIENTIFIQUE, Paris (FR); INSTITUT NATIONAL DE LA SANTÉ ET DE LA RECHERCHE MÉDICALE, Paris (FR)

(21) Appl. No.: 18/693,250

(22) PCT Filed: Oct. 7, 2022
(86) PCT No.: PCT/EP2022/077890

§ 371 Date: Mar. 19, 2024

(30) Foreign Application Priority Data
Oct. 12, 2021 (FR) FR2110762

(51) Int. Cl. C07K 1/22; C07K 14/39

(52) U.S. Cl. CPC C07K 1/22; C07K 14/39; C07K 2319/20

(57) ABSTRACT

A method for purifying a protein of interest includes the preparation of a fusion protein in which this protein of interest is fused to a protein tag having the protein Mmi1 of a microorganism of the genus Schizosaccharomyces or a fragment thereof, the bringing into contact of this fusion protein with a ribonucleic acid molecule containing at least one motif of UNAAAC nucleotide sequence, so as to allow the affinity binding of the protein tag with this ribonucleic acid molecule, and the recovery of the protein of interest.

### REFERENCE TO AN ELECTRONIC SEQUENCE LISTING

The present application contains a Sequence Listing that has been submitted electronically and is hereby incorporated by reference in its entirety. The electronic Sequence Listing is named Sequence_Listing.xml, was created on Sep. 11, 2024, and is 67,950 bytes in size.

The present invention falls within the field of affinity purification of the proteins of interest.

More particularly, the present invention concerns a method for purifying a protein of interest. Other objects of the invention are a solid support and a kit for implementing a purification method according to the invention.

The production in large quantities and at low cost of proteins of interest is of growing interest in many sectors of the biotechnology industry.

At present, the proteins are mainly produced by culturing cell lines specially designed to express them from the genes encoding these proteins, which are integrated into these cell lines. If such biological production methods make it possible to produce the proteins of interest efficiently and in large quantities, the latter are however obtained within complex mixtures, containing in particular the elements necessary for the culture of the cell lines, as well as other cell components of the cells. It is therefore necessary to isolate the proteins of interest produced from these complex mixtures, in order to recover them in a sufficiently pure form for the targeted applications, this having to be carried out without compromising the biological activity necessary for these applications. This objective is particularly crucial when the proteins of interest are, for example, hormones, antibiotic peptides, enzyme regulators, etc., or any other protein intended for a therapeutic application.

Numerous methods have been proposed by the prior art for the purification of the proteins of interest produced by biological methods.

Many of these methods consist of expressing the protein of interest in the form of a fusion protein in which it is associated, by the genetic engineering techniques, with a protein tag having a particular affinity for a partner. It is then possible to purify the protein of interest by affinity chromatography, the partner of the protein tag being grafted onto the chromatography support.

As such protein tags proposed by the prior art, we may in particular cite the histidine tag, having a high affinity for the cobalt and nickel cations, the Glutathione S-transferase tag, having a high affinity for the glutathione, and the maltose binding protein (MBP), with high affinity for the maltose.

As another example, document WO 2017/194888 describes a method for affinity purifying proteins of interest based on the lectin activity of the CRD domain of a galectin.

None of the methods proposed by the prior art, however, makes it possible to produce and purify a protein of interest both at low cost, quickly, and with high yield and high specificity leading to obtaining the protein of interest with a high purity rate. The present invention aims to provide such a method.

To this end, the present invention takes advantage of the strong capacity of a particular domain of the protein Mmi1 of species of the genus Schizosaccharomyces to bind to a ribonucleic acid (RNA) molecule of particular sequence, this with high specificity.

The protein Mmi1 (Meiotic mRNA interception protein 1) plays an important role within cells in a particular post-transcriptional event, the selective elimination of meiosis-specific messenger RNAs. It has been described in the literature that this protein binds with high RNA binding specificity, more particularly to a sequence containing repeats of the hexanucleotide UNAAAC (E. Hiriart et al., The Embo Journal, 2012, 31(10), 2296-2308; Wu et al., Biochemical and Biophysical Research Communications, 2017, 491, 310-316). This affinity has been more particularly attributed to the domain called YTH (for YT521-B homology) of Mmi1 of Schizosaccharomyces, located in the C-terminal region of the protein (Stowell et al., J. Biol. Chem., 2018, 293(24), 9210-9222).

The publication by Shichino Yuichi et al., in Plos Genetics, 2020, 16(2):e1008598 describes a fusion protein between rrp6, GFP or YFP and the protein Mmi1 of whole or truncated Schizosaccharomyces pombe. The publication by Xie Guodong et al., in Nature Communications, 2019, 10:251 describes a fusion protein between Erh1 or GST and Mmi1 of whole or truncated Schizosaccharomyces pombe.

Quite surprisingly, it was discovered by the present inventors that not only the fusion of a protein Mmi1 of species of the genus Schizosaccharomyces, or of one of its fragments comprising at least its YTH domain extended on the N-terminal side and on the C-terminal side (this expanded YTH domain, constituted by the 173 C-terminal amino acids of the protein Mmi1, being designated in the present description, for convenience, by the abbreviation “YTH+”), to a protein of interest, does not impact the ability of this protein Mmi1 or of this fragment to bind specifically to the RNA sequence with UNAAAC motif mentioned above, whether the fusion is carried out at the N-terminal or at the C-terminal of the protein Mmi1 or of its fragment; but in addition, the bringing of a ribonucleic acid molecule containing at least one UNAAAC nucleotide sequence motif into contact with a cell culture extract containing a fusion protein “protein of interest/protein Mmi1 or fragment of this last containing the YTH+ domain”, does not cause massive degradation of this RNA molecule in this cell culture extract. Thus, while those skilled in the art would never have considered implementing, in a method for purifying a protein of interest produced by a biological method, a step of bringing the medium containing the protein to be purified into contact with a RNA molecule as a partner for affinity binding to the tag fused to the protein of interest, it has been discovered by the present inventors that not only is such a method entirely feasible for the particular case of the pair “Mmi1 of Schizosaccharomyces or fragment of the latter containing the YTH+ domain/RNA molecule with UNAAAC sequence”, but that it also proves to be highly efficient, since it makes it possible to purify the protein of interest with high specificity and high yield, in a short time, without altering the functions of the protein of interest.

Thus, according to a first aspect, the present invention proposes a method for purifying a protein of interest, which comprises:
the preparation of a fusion protein comprising the protein of interest fused to a protein tag, said protein tag comprising at least, or consisting of: the protein Mmi1 of a microorganism of the genus Schizosaccharomyces, a fragment of this protein Mmi1 comprising at least the 173 C-terminal amino acids (the domain of the protein Mmi1 formed by these 173 C-terminal amino acids being designated in the present description by the abbreviation YTH+), or a protein of amino acid sequence having at least 90% identity with the amino acid sequence of said protein Mmi1 or said fragment and capable of binding to a ribonucleic acid motif of UNAAAC nucleotide sequence,
the bringing of this fusion protein into contact with a ribonucleic acid molecule containing at least one motif of UNAAAC nucleotide sequence, so as to allow the affinity binding of the protein tag with this ribonucleic acid molecule, more precisely with its motif of UNAAAC nucleotide sequence, this ribonucleic acid molecule being grafted onto a solid support or coupled to a capture ligand,
where appropriate, when said ribonucleic acid molecule is coupled to a capture ligand, the bringing of this ribonucleic acid molecule, to which the protein of interest is bound through the protein tag, into contact with an affinity partner of the capture ligand grafted onto a solid support,
optionally, the isolation of the solid support from the medium in which it is present, for example a cell culture medium in which the cells expressing the fusion protein have been cultured, and/or a cell lysis medium for such cells,
and the separation of the protein of interest, alone or within the fusion protein which contains it, and from the solid support, to recover the protein of interest.

In the present description, the term capture ligand means a molecule which is on the one hand capable of being covalently coupled to a ribonucleic acid molecule, and on the other hand capable of binding with high affinity and specificity to an affinity partner, for example a protein receptor, the latter being able to be attached to a solid support. As an example of such a capture ligand which can be used according to the invention, mention may be made of biotin, the affinity partners of which are avidin and streptavidin.

The method according to the invention advantageously makes it possible, on its own, to produce the protein of interest, in the form of a recombinant fusion protein, and to separate it from the production medium by taking advantage of the strong, highly specific binding capacity of the protein tag comprising the YTH+ domain of a protein Mmi1 of a species of the genus Schizosaccharomyces with the UNAAAC RNA sequence, to obtain the protein of interest quickly with a high degree of purity and a high yield. These steps may advantageously be carried out easily and quickly, and at low cost.

Conventionally, in the UNAAAC nucleotide sequence, U designates uracil, A designates adenine, C designates cytosine and N designates any base among adenine, cytosine, guanine and uracil. This nucleotide sequence is represented here, like all the other nucleotide sequences described, conventionally, that is to say in the direction from the 5′ end to the 3′ end (the amino acid sequences being as to them represented, also conventionally, in the reading direction from the N terminus to the C terminus).

The ribonucleic acid molecule containing the UNAAAC motif may comprise a single copy of this motif, or one or more repeats of this motif. It may further comprise, at 5′ or at 3′ of this UNAAAC motif or of this series of UNAAAC motifs, one or more additional ribonucleic acids, for example 2 to 10, in particular 2 to 6, additional ribonucleic acids.

The grafting/coupling of the ribonucleic acid molecule to the solid support/to the capture ligand may be carried out by any method conventional in itself. This grafting/coupling is preferably carried out by covalent bond, preferably at the 5′ end or at the 3′ end of the ribonucleic acid molecule.

In the present description, protein of interest means any protein, peptide or polypeptide, in native or recombinant form, of interest for a targeted application, in particular for an application comprising administration to a mammal, in particular a human.

The method according to the invention may, for example, advantageously be used for the purification of endogenous complexes in the field of fundamental research, of hormones, of antibiotic peptides or even of enzyme regulators in the field of applied research, such a list is in no way limiting to the invention.

The protein tag implemented in the method according to the invention may comprise the protein Mmi1 of a microorganism of a species of the whole genus Schizosaccharomyces or one of its fragments containing at least the YTH+ domain (that is to say the 173 C-terminal amino acids).

The protein Mmi1 preferably comes from a species chosen from Schizosaccharomyces pombe, Schizosaccharomyces japonicus, Schizosaccharomyces octosporus and Schizosaccharomyces cryophilus.

It is the responsibility of those skilled in the art to identify, for a given species of the genus Schizosaccharomyces, the amino acid sequence of the protein Mmi1, as well as, where appropriate, the sequence of the gene encoding this protein. Such data are particularly accessible in protein sequence and nucleotide sequence databases. For example, in the GenBank database, the amino acid sequences of the protein Mmi1 are accessible, for the species Schizosaccharomyces pombe, under the accession number NP_587783.2 (SEQ ID No: 1—the gene encoding this protein of this species has the sequence SEQ ID No: 2), for the species Schizosaccharomyces japonicus, under the accession number XP_002173827.2 (SEQ ID No: 3—the gene encoding this protein of this species has the sequence SEQ ID No: 4), for the species Schizosaccharomyces octosporus, under the accession number XP_013019124.1 (SEQ ID No: 5—the gene encoding this protein of this species has the sequence SEQ ID No: 6), and for the species Schizosaccharomyces cryophilus, under the accession number XP_013025346.1 (SEQ ID No: 7—the gene encoding this protein of this species has the sequence SEQ ID No: 8).

When the microorganism of the genus Schizosaccharomyces belongs to the species Schizosaccharomyces pombe, the YTH+ domain of the protein Mmi1 extends from the residue at position 316 (leucine residue) to the residue at position 488 (arginine residue, at the C-terminal position in the sequence of the protein). The YTH+ domain then has as the amino acid sequence the sequence SEQ ID No: 9.

When the microorganism of the genus Schizosaccharomyces belongs to one of the species Schizosaccharomyces japonicus, Schizosaccharomyces octosporus and Schizosaccharomyces cryophilus, the fragment of the protein Mmi1 preferably contains the 174 C-terminal amino acids of the protein. The fragment of the protein Mmi1 used according to the invention thus preferably contains, or consists of:
for the species Schizosaccharomyces japonicus, the domain of the protein Mmi1 extending from the residue at position 306 (leucine residue) to the residue at position 479 (arginine residue, at the C-terminal position in the sequence of the protein). The domain then has as the amino acid sequence the sequence SEQ ID No: 10;
for the species Schizosaccharomyces octosporus, the domain of the protein Mmi1 extending from the residue at position 307 (leucine residue) to the residue at position 480 (arginine residue, at the C-terminal position in the sequence of the protein). The domain then has as the amino acid sequence the sequence SEQ ID No: 11;
for the species Schizosaccharomyces cryophilus, the domain of the protein Mmi1 extending from the residue at position 306 (leucine residue) to the residue at position 479 (arginine residue, at the C-terminal position in the sequence of the protein). The domain then has as the amino acid sequence the sequence SEQ ID No: 12.

Thus, in particular embodiments of the invention, the fragment of the protein Mmi1 comprising at least the 173 C-terminal amino acids has an amino acid sequence comprising, or consisting of, the acid sequence amines SEQ ID No: 9 (corresponding to Schizosaccharomyces pombe), SEQ ID No: 10 (corresponding to Schizosaccharomyces japonicus), SEQ ID No: 11 (corresponding to Schizosaccharomyces octosporus) or SEQ ID No: 12 (corresponding to Schizosaccharomyces cryophilus):

SEQ ID No: 9:
LNFSRASEHRNEKGERISMINPRVVLDENGISHRSRYFIMLCDNETAIA
HAKKTSIWAVKKDSSKRISDAYKKASVYFIFVAQQTYNALGYAQVVSDL
NSTELPFWSDSSHAGGVRIKWIKTCNLFSAEISEIVSHMDHGSEARDGM
EMMYDEGSRLCTLINYAIMKRIGRDR
SEQ ID No: 10:
LDFEHAHEYRNEKGERVSMINPRVILDENGISHRSRYFIMLVDNETAIS
HAKRSSVWSLKQNYANAISEAYKQEANVYFIFINAKSYNALGYAQLTSD
VGTVAKPFWADTTYTNGVNVKWIKTCNLYSNEISEIVSRMDHGARARDG
QEMMYDEGSRLCTLINSAIMKRIGRDR
SEQ ID No: 11:
LNFARASEYRNDKGERISMINPRVILDENGISSRSRYFIMLCDNETAIS
HAKKTSIWAVKHDAASRVSDAYKNASIYFIFIAKPTNNALGYAQVVSDL
NSAELPFWADNATYAGGVRVKWIKTCNLFSAEISDIVGRMNHGATARDG
MEMMYDEGCRLCILVNSAIMKRIGRDR
SEQ ID No: 12:
LNFARASEYRNDKGERISMINPRVILDESGISHRSRYFIMLCDNETAIA
HAKKTSIWAVKHEAASRVSDAYKKASIYFIFIAKPTNNALGYAQVVSDL
NSAELPFWADNATYAGGVRVKWIKTCNLFSAEISDIVGRMNHGMTAKDG
MEMMYDEGCRLCILVNSAIMKRIGRDR

The protein tag implemented according to the invention may as well comprise, or consist of, the protein Mmi1 or a fragment of the protein Mmi1 containing at least the YTH+ domain of the protein, as well as a protein of amino acid sequence having at least 90%, preferably at least 95%, preferentially at least 98% and more preferentially at least 99%, identity with the amino acid sequence of the protein Mmi1 or of said fragment of said protein Mmi1, and capable of binding to a ribonucleic acid motif of UNAAAC nucleotide sequence, preferably with a specificity at least as good as the YTH+ domain of the protein.

For any protein of given amino acid sequence having at least 90%, preferably at least 95%, preferentially at least 98% and more preferentially at least 99%, identity with the amino acid sequence of the protein Mmi1 or of said fragment of the protein Mmi1, it is within the skills of those skilled in the art to evaluate its binding capacity to the RNA motif of UNAAAC sequence, by binding assays conventional in themselves, for example by gel mobility shift assays after electrophoresis or fluorescence spectroscopy, circular dichroism or plasmon resonance. The binding specificity may be evaluated by these same methods, by comparing with RNA molecules of sequence close to the UNAAAC sequence (for example the CNAAAC or GNAAAC sequence) and comparing the results obtained with those obtained with said protein Mmi1 or said fragment of the protein Mmi1.

The protein of amino acid sequence having at least 90%, preferably at least 95%, preferentially at least 98% and more preferentially at least 99%, identity with the amino acid sequence of the protein Mmi1 or of said fragment of the protein Mmi1 may have, relative to the sequence of the protein Mmi1 or of said fragment of the protein Mmi1, which constitutes the reference sequence, insertions, deletions and/or substitutions. In the case of a substitution, this is preferably carried out by an amino acid of the same family as the original amino acid, for example by substitution of a basic residue such as the arginine by another basic residue such as a lysine residue, of an acidic residue such as the aspartate by another acidic residue such as the glutamate, of a polar residue such as the serine by another polar residue such as the threonine, of an aliphatic residue such as the leucine by another aliphatic residue such as the isoleucine, etc.

The percentage of identity between two amino acid sequences is here determined in a conventional manner in itself, by comparing the two optimally aligned sequences, through a comparison window, the part of the amino acid sequence to be compared located in the comparison window which may comprise additions or deletions relative to the reference sequence so as to obtain an optimal alignment between the two sequences. The percentage of identity is then calculated by determining the number of positions for which an amino acid residue is identical in the two compared sequences, then dividing that number of positions by the total number of positions in the comparison window, the obtained number being multiplied by one hundred to obtain the percentage of identity between the two sequences.

The method according to the invention may also meet one or more of the characteristics described below, implemented individually or in each of their technically effective combinations.

Within the fusion protein, the protein tag may be fused to the N terminus or the C terminus of the protein of interest.

If necessary, a spacer may be integrated between the protein of interest and the protein tag.

In particular embodiments of the invention, an enzymatic cleavage site is inserted between the protein of interest and the protein tag. Any cleavage site conventional in itself falls within the scope of the invention, in particular the protease cleavage sites such as the cleavage site of the TEV protease (tobacco etch virus protease).

In such embodiments, the method according to the invention preferably comprises a step of cleaving the fusion protein, by a suitable enzyme, at this enzymatic cleavage site, so as to separate the protein of interest from the protein tag.

Such a step of cleaving the fusion protein is however only optional, and is in particular only necessary when the fusion of the protein tag to the protein of interest modifies the activity of the latter useful for the intended application of the protein of interest. When the fusion of the protein tag to the protein of interest does not modify the activity of the latter, and does not induce any harmful side effects in the context of the intended application, then a step of separating the protein of interest and the protein tag is not necessary. In particular, the protein Mmi1s of the genus Schizosaccharomyces interact with RNA in an entirely different manner from mammalian YTH domain proteins, so that the fusion protein prepared and purified according to the invention may advantageously be administered, as is, to a mammal, in particular to a human, without the protein tag disrupting normal biological processes.

Quite advantageously, the presence of the protein tag according to the invention in fusion with the protein of interest does not cause any undesirable effects in the higher eukaryotic cells. This protein tag does not have any toxicity for these cells, and does not cause any modification in the localization of the protein of interest.

The step of preparing the fusion protein may be carried out in any conventional manner, in particular by biological means, by implementing genetic engineering techniques conventional in themselves.

In particular, the fusion protein may be prepared by transfecting a suitable host organism with a nucleic acid molecule encoding the fusion protein, or transforming a suitable host organism with an expression vector in which the hybrid gene encoding the fusion protein is operably bound to a DNA sequence controlling its expression; and culturing this host organism under conditions allowing the expression of the fusion protein, such conditions being conventional in themselves and well known to those skilled in the art.

The host organisms that can be used for this purpose include, but are not limited to, gram-positive and gram-negative bacteria such as strains of Escherichia coli or Bacillus subtilis, yeasts such as strains of Saccharomyces cerevisiae, and higher eukaryotes organisms, including mammalian cell lines.

The hybrid gene encoding the fusion protein may be prepared by conventional DNA recombination methods or by gene synthesis methods also conventional in themselves. It may be incorporated into any conventional protein expression vector.

At the end of this preparation step of the method according to the invention, the produced fusion protein is separated from the host organism and the culture medium by bringing it into contact with the medium containing it, if necessary after a cell lysis (such a cell lysis step not being necessary when the fusion protein to be separated from the medium is of the extracellular addressing type) the target RNA molecule, grafted onto a solid support or covalently coupled to a capture ligand.

In particular embodiments of the invention, this ribonucleic acid molecule contains one or more repeats of the UNAAAC sequence motif. These different repeats may either be contiguous or spaced from each other by a spacer sequence.

The ribonucleic acid molecule containing at least one UNAAAC nucleotide sequence motif may comprise, within this motif and/or in any other position:
at least one chemically modified nucleotide, in particular a 2′-O-methylated nucleotide, such a modification being carried out in a conventional manner in itself, in particular at the ribose motif;
and/or at least one locked nucleic acid, called LNA, that is to say a nucleic acid analogue containing a methylene bridge between the hydroxyl at position 2 and the atom at position 4 of the sugar;
and/or at least one phosphorothiate internucleotide bond.

The step of bringing the fusion protein into contact with the ribonucleic acid molecule containing at least one UNAAAC nucleotide sequence motif is preferably carried out for a period comprised between 5 minutes and 1 hour, preferably comprised between 10 minutes and 30 minutes.

The solid support on which the affinity partner of the capture ligand or the ribonucleic acid molecule is grafted may be of any type conventional in itself, in particular for carrying out immunoprecipitation separation methods. It may for example consist of agarose or polystyrene beads, for example with magnetic properties, which can in particular be isolated from the medium containing them by magnets.

In particular embodiments of the invention, the solid support is a chromatography support, conventional in itself, for example a crosslinked polymer based on polysaccharide, such as the dextran or the agarose, or polyacrylamide, particularly in the form of porous beads, or polystyrene.

For the implementation of the method according to the invention, the chromatography support is then preferably present in a column (or tube), into which is introduced the medium containing the fusion protein, where appropriate bound to the ribonucleic acid molecule when the method according to the invention implements a capture ligand coupled to the latter, for the attachment of the fusion protein to the chromatography support according to the principle of the affinity chromatography, this affinity being either between the capture ligand and its affinity partner grafted onto the support, or between the fusion protein, more precisely the protein tag, and the ribonucleic acid molecule grafted onto the support.

Such a chromatography column may be operated in batch or continuous mode.

Preferably, prior to its bringing into contact with the fusion protein, the chromatography support is equilibrated, in a conventional manner in itself and according to the manufacturer's recommendations, in particular by an aqueous equilibration buffer. This equilibration buffer may contain a denaturing agent or a detergent such as the guanidinium chloride, the urea or the Triton® X-100.

The separation of the fusion protein and the solid support may be carried out in different ways, all comprising a step of eluting the protein of interest (alone or in the form of fusion protein with the protein tag). This elution may be carried out at constant pH or with pH gradients decreasing linearly or discontinuously. The optimal elution conditions are easily determinable by those skilled in the art, by routine experiments, for each given protein of interest.

The elution buffer may contain a denaturing agent or a detergent such as the guanidinium chloride, the urea or the Triton® X-100. The addition of such a denaturing agent or detergent facilitates the purification, even when the protein of interest is poorly soluble in aqueous solution.

In particular embodiments of the invention in which an enzymatic cleavage site is inserted between the protein of interest and the protein tag, the separation of the protein of interest and the solid support may be carried out by cleavage at the enzymatic cleavage site. The protein of interest may then be recovered by elution, as indicated above.

In variants of the invention, the fusion protein may be separated from the solid support by chemical elution, in particular by means of a glycine-based solution of low pH, then the protein of interest may optionally be dissociated from the protein tag by cleavage at the enzymatic cleavage site that separates them.

In particular embodiments of the invention, a cleavage site is inserted between the ribonucleic acid molecule containing at least one UNAAAC nucleotide sequence motif and the solid support or the capture ligand. This cleavage site may be of the type cleavable by a specific RNAse enzyme. In such a configuration, the separation of the protein of interest and the chromatography support may be carried out by cleavage at this cleavage site. The fusion protein may then be recovered by elution, as indicated above.

The separation of the fusion protein and of the solid support may otherwise be carried out by chemical cleavage within the immobilized RNA molecule, directly or indirectly, through the “capture ligand/affinity partner” couple, on the solid support.

In particular embodiments of the invention, the protein tag contains, in addition to the fragment of the protein Mmi1 containing the 173 or 174 C-terminal amino acids, at least one domain chosen from the following domains of Mmi1 of Schizosaccharomyces of amino acid sequence:
RSVWXaa₁Xaa₂Xaa₃Xaa₄Xaa₅Xaa₆P (SEQ ID No: 13) (domain 2 of Mmi1 of Schizosaccharomyces), where Xaa₁ represents a threonine, serine or alanine residue, Xaa₂ represents a threonine, arginine, lysine or serine residue, Xaa₃ represents a histidine or arginine residue, Xaa₄ represents a threonine or proline residue, Xaa₅ represents a glycine, alanine or arginine residue and Xaa₆ represents a glutamic acid or aspartic acid residue, this domain being designated in the present description as the “domain 2”;
FXaa₇SPLKRXaa₈APXaa₉SXaa₁₀Xaa₁₁Xaa₁₂Xaa₁₃Xaa₁₄Xaa₁₅R (SEQ ID No: 14) (domain 3 of Mmi1 of Schizosaccharomyces), where Xaa₇ represents a serine or threonine residue, Xaa₈ represents a proline or glycine residue, Xaa₉ represents a glutamic acid or aspartic acid residue, Xaa₁₀ represents a histidine, arginine or lysine residue, Xaa₁₁ represents an aspartic acid or glutamic acid residue, Xaa₁₂ represents an alanine or tyrosine residue, Xaa₁₃ is zero or represents a proline residue, Xaa₁₄ represents an isoleucine or methionine residue and Xaa₁₅ represents a glycine or aspartic acid residue, this domain being designated in the present description as the “domain 3”;
YDFXaa₁₆RHCTDYGHSYXaa₁₇WPYFRSXaa₁₈RREXaa₁₉Xaa₂₀Xaa₂₁Y (SEQ ID No: 15) (domain 4 of Mmi1 of Schizosaccharomyces), where Xaa₁₆ represents a serine, threonine or tyrosine residue, Xaa₁₇ represents a glutamic acid or aspartic acid residue, Xaa₁₈ represents a leucine or valine residue, Xaa₁₉ is zero or represents a serine residue, Xaa₂₀ represents a leucine or methionine residue and Xaa₂₁ represents an arginine, leucine or methionine residue, this domain being designated in the present description as the “domain 4”.
QPPXaa₂₂KRRTLXaa₂₃Xaa₂₄P (SEQ ID No: 16) (domain 5 of Mmi1 of Schizosaccharomyces), where Xaa₂₂ represents a proline, serine or leucine residue, Xaa₂₃ represents a serine or leucine residue and Xaa₂₄ represents a proline or serine residue, this domain being designated in the present description as the “domain 5”;

(SEQ ID NO: 17)
Xaa₂₅AXaa₂₆Xaa₂₇SPXaa₂₈Xaa₂₉Xaa₃₀Xaa₃₁PXaa₃₂Xaa₃₃H

(domain 6 of Mmi1 of Schizosaccharomyces), where Xaa₂₅ represents an arginine or aspartic acid residue, Xaa₂₆ represents a serine or glycine residue, Xaa₂₇ represents a histidine or aspartic acid residue, Xaa₂₈ represents a serine, glycine or leucine residue, Xaa₂₉ represents a leucine or phenylalanine residue, Xaa₃₀ represents a leucine, isoleucine or serine residue, Xaa₃₁ represents a glutamic acid or aspartic acid residue, Xaa₃₂ represents a tyrosine or threonine residue and Xaa₃₃ represents an alanine or threonine residue, this domain being designated in the present description as the “domain 6”;
RXaa₃₄EKPKXaa₃₅RAXaa₃₆TPPP (SEQ ID No: 18) (domain 7 of Mmi1 of Schizosaccharomyces), where Xaa₃₄ represents a lysine or arginine residue, Xaa₃₅ represents an alanine, proline or threonine residue and Xaa₃₆ represents a serine or proline residue, this domain being designated in the present description as the “domain 7”.

The protein tag may contain two or more of the domains 2 to 7 above, including all of these domains, any combination of two or more of these domains falling within the scope of the invention.

Preferably, in the amino acid sequence of each of the domains 2 to 7 above, the variable amino acids are chosen so that the amino acid sequence of each of the domains corresponds to the amino acid sequence of a domain of the protein Mmi1 of a microorganism of the genus Schizosaccharomyces, preferably the same as that from which said fragment containing the YTH+ domain of the protein is derived. The different domains, as well as the YTH+ domain, contained in the protein tag according to the invention are then preferably positioned relative to each other according to their normal positioning in the native protein, these domains then being contiguous or separated by the native intron sequences of the protein, or by sequences obtained by substitution, addition or deletion with respect to these native intron sequences.

In particular embodiments of the invention, the protein tag comprises the YTH+ domain of the protein Mmi1 of Schizosaccharomyces pombe and at least one domain of this protein chosen from the following domains, or several of these domains, or even the entirety, according to all possible combinations:
domain 2: extending from the amino acid at position 40 (arginine) to the amino acid at position 50 (proline) of the protein Mmi1, of sequence: RSVWTTHTGEP (SEQ ID No: 19), or a protein region of amino acid sequence having at least 90%, preferably at least 95%, more preferably at least 98%, and preferentially at least 99%, identity with this amino acid sequence;
domain 3: extending from the amino acid at position 64 (phenylalanine) to the amino acid at position 82 (arginine) of the protein Mmi1, of sequence: FSSPLKRPAPESHDAPIGR (SEQ ID No: 20), or a protein region of amino acid sequence having at least 90%, preferably at least 95%, more preferably at least 98%, and preferentially at least 99%, identity with this amino acid sequence;
domain 4: extending from the amino acid at position 97 (tyrosine) to the amino acid at position 125 (tyrosine) of the protein Mmi1, of sequence: YDFSRHCTDYGHSYEWPYFRSLRRESMLY (SEQ ID No: 21), or a protein region of amino acid sequence having at least 90%, preferably at least 95%, more preferably at least 98%, and preferentially at least 99%, identity with this amino acid sequence;
domain 5: extending from the amino acid at position 169 (glutamine) to the amino acid at position 180 (proline) of the protein Mmi1, of sequence: QPPPKRRTLSPP (SEQ ID No: 22), or a protein region of amino acid sequence having at least 90%, preferably at least 95%, more preferably at least 98%, and preferentially at least 99%, identity with this amino acid sequence;
domain 6: extending from the amino acid at position 258 (arginine) to the amino acid at position 272 (histidine) of the protein Mmi1, of sequence: RASHSPSLLEPYAH (SEQ ID No: 23), or a protein region of amino acid sequence having at least 90%, preferably at least 95%, more preferably at least 98%, and preferentially at least 99%, identity with this amino acid sequence;
domain 7: extending from the amino acid at position 302 (arginine) to the amino acid at position 315 (proline) of the protein Mmi1, of sequence: RKEKPKARASTPPP (SEQ ID No: 24), or a protein region of amino acid sequence having at least 90%, preferably at least 95%, more preferably at least 98%, and preferentially at least 99%, identity with this amino acid sequence.

As indicated above, these different domains, as well as the YTH+ domain, contained in the protein tag, are preferably positioned relative to each other according to their normal positioning in the native protein, these domains then being contiguous or separated by the native intron sequences of the protein, or by sequences obtained by substitution, addition or deletion with respect to these native intron sequences.

In particular embodiments of the invention, the protein tag comprises, or consists of, the protein Mmi1 of Schizosaccharomyces pombe deleted of the 30 amino acids at N-terminal position. The protein tag then comprises, or consists of, the amino acid sequence of sequence SEQ ID No: 25.

In particular embodiments of the invention, the protein tag comprises the YTH+ domain of the protein Mmi1 of Schizosaccharomyces japonicus and at least one domain of this protein chosen from the following domains, or several of these domains, or even the entirety, according to all possible combinations:
domain 2: extending from the amino acid at position 38 (arginine) to the amino acid at position 48 (proline) of the protein Mmi1, of sequence: RSVWAKHPNDP (SEQ ID No: 26), or a protein region of amino acid sequence having at least 90%, preferably at least 95%, more preferably at least 98%, and preferentially at least 99%, identity with this amino acid sequence;
domain 3: extending from the amino acid at position 61 (phenylalanine) to the amino acid at position 78 (arginine) of the protein Mmi1, of sequence: FSSPLKRGAPDSKEYMDR (SEQ ID No: 27), or a protein region of amino acid sequence having at least 90%, preferably at least 95%, more preferably at least 98%, and preferentially at least 99%, identity with this amino acid sequence;
domain 4: extending from the amino acid at position 93 (tyrosine) to the amino acid at position 120 (tyrosine) of the protein Mmi1, of sequence: YDFYRHCTDYGHSYDWPYFRSLRRELAY (SEQ ID No: 28), or a protein region of amino acid sequence having at least 90%, preferably at least 95%, more preferably at least 98%, and preferentially at least 99%, identity with this amino acid sequence;
domain 5: extending from the amino acid at position 166 (glutamine) to the amino acid at position 177 (proline) of the protein Mmi1, of sequence: QPPLKRRTLLSP (SEQ ID No: 29), or a protein region of amino acid sequence having at least 90%, preferably at least 95%, more preferably at least 98%, and preferentially at least 99%, identity with this amino acid sequence;
domain 6: extending from the amino acid at position 245 (aspartic acid) to the amino acid at position 258 (histidine) of the protein Mmi1, of sequence: DAGDSPLFSEPTAH (SEQ ID No: 30), or a region protein of amino acid sequence having at least 90%, preferably at least 95%, more preferably at least 98%, and preferentially at least 99%, identity with this amino acid sequence;
domain 7: extending from the amino acid at position 292 (arginine) to the amino acid at position 305 (proline) of the protein Mmi1, of sequence: RREKPKTRAPTPPP (SEQ ID No: 31), or a protein region of amino acid sequence having at least 90%, preferably at least 95%, more preferably at least 98%, and preferentially at least 99%, identity with this amino acid sequence.

In alternative embodiments of the invention, the protein tag comprises the YTH+ domain of the protein Mmi1 of Schizosaccharomyces octosporus and at least one domain of this protein chosen from the following domains, or several of these domains, or even the entirety, according to all possible combinations:
domain 2: extending from the amino acid at position 39 (arginine) to the amino acid at position 49 (proline) of the protein Mmi1, of sequence: RSVWSNRPAEP (SEQ ID No: 32), or a protein region of amino acid sequence having at least 90%, preferably at least 95%, more preferably at least 98%, and preferentially at least 99%, identity with this amino acid sequence;
domain 3: extending from the amino acid at position 61 (phenylalanine) to the amino acid at position 79 (arginine) of the protein Mmi1, of sequence: FTSPLKRPAPDSREAPMGR (SEQ ID No: 33), or a protein region of amino acid sequence having at least 90%, preferably at least 95%, more preferably at least 98%, and preferentially at least 99%, identity with this amino acid sequence;
domain 4: extending from the amino acid at position 94 (tyrosine) to the amino acid at position 122 (tyrosine) of the protein Mmi1, of sequence: YDFTRHCTDYGHSYEWPYFRSVRRESLMY (SEQ ID No: 34), or a protein region of amino acid sequence having at least 90%, preferably at least 95%, more preferably at least 98%, and preferentially at least 99%, identity with this amino acid sequence;
domain 5: extending from the amino acid at position 170 (glutamine) to the amino acid at position 181 (proline) of the protein Mmi1, of sequence: QPPSKRTLSPP (SEQ ID No: 35) or a protein region of amino acid sequence having at least 90%, preferably at least 95%, more preferably at least 98%, and preferentially at least 99%, identity with this amino acid sequence;
domain 6: extending from the amino acid at position 253 (arginine) to the amino acid at position 266 (histidine) of the protein Mmi1, of sequence: RASHSPGLIDPYTH (SEQ ID No: 36), or a protein region of amino acid sequence having at least 90%, preferably at least 95%, more preferably at least 98%, and preferentially at least 99%, identity with this amino acid sequence;
domain 7: extending from the amino acid at position 293 (arginine) to the amino acid at position 306 (proline) of the protein Mmi1, of sequence: RKEKPKPRAPTPPP (SEQ ID No: 37), or a protein region of amino acid sequence having at least 90%, preferably at least 95%, more preferably at least 98%, and preferentially at least 99%, identity with this amino acid sequence.

In still alternative embodiments of the invention, the protein tag comprises the YTH+ domain of the protein Mmi1 of Schizosaccharomyces cryophilus and at least one domain of this protein chosen from the following domains, or several of these domains, or even the entirely, according to all possible combinations:
domain 2: extending from the amino acid at position 39 (arginine) to the amino acid at position 49 (proline) of the protein Mmi1, of sequence: RSVWSSRPAEP (SEQ ID No: 38), or a protein region of amino acid sequence having at least 90%, preferably at least 95%, more preferably at least 98%, and preferentially at least 99%, identity with this sequence amino acids;
domain 3: extending from the amino acid at position 61 (phenylalanine) to the amino acid at position 79 (proline) of the protein Mmi1, of sequence: FTSPLKRPAPDSREAPIGR (SEQ ID No: 39), or a protein region of amino acid sequence having at least 90%, preferably at least 95%, more preferably at least 98%, and preferentially at least 99%, identity with this amino acid sequence;
domain 4: extending from the amino acid at position 94 (tyrosine) to the amino acid at position 122 (tyrosine) of the protein Mmi1, of sequence: YDFTRHCTDYGHSYEWPYFRSVRRESLMY (SEQ ID No: 40), or a protein region of amino acid sequence having at least 90%, preferably at least 95%, more preferably at least 98%, and preferentially at least 99%, identity with this amino acid sequence;
domain 5: extending from the amino acid at position 169 (glutamine) to the amino acid at position 180 (proline) of the protein Mmi1, of sequence: QPPSKRRTLSPP (SEQ ID No: 41) or a protein region of amino acid sequence having at least 90%, preferably at least 95%, more preferably at least 98%, and preferentially at least 99%, identity with this amino acid sequence;
domain 6: extending from the amino acid at position 252 (aspartic acid) to the amino acid at position 265 (histidine) of the protein Mmi1, of sequence: RASHSPSLIDPYAH (SEQ ID No: 42), or a protein region of amino acid sequence having at least 90%, preferably at least 95%, more preferably at least 98%, and preferentially at least 99%, identity with this amino acid sequence;
domain 7: extending from the amino acid at position 292 (arginine) to the amino acid at position 305 (proline) of the protein Mmi1, of sequence: RKEKPKPRAPTPPP (SEQ ID No: 43), or a protein region of amino acid sequence having at least 90%, preferably at least 95%, more preferably at least 98%, and preferentially at least 99%, identity with this amino acid sequence.

The fusion protein according to the invention may also comprise other chemical or protein tags, conventional in themselves, one or more subcellular localization sequences, etc.

A recombinant fusion protein, capable of being obtained at the end of the step of preparing a fusion protein of the purification method according to the invention, or at the end of the method according to the invention, in purified form, comprises a protein of interest fused to a protein tag, this protein tag comprising at least, or consisting of: the protein Mmi1 of a microorganism of the genus Schizosaccharomyces, a fragment of this protein Mmi1 comprising at least the 173 C-terminal amino acids, or a protein of amino acid sequence having at least 90% identity, preferably at least 95%, preferentially at least 98%, and more preferentially at least 99% identity with the amino acid sequence of said protein Mmi1 or of said fragment and capable of binding to a ribonucleic acid motif of UNAAAC nucleotide sequence.

This fusion protein may have any characteristic or combination of characteristics described above with reference to the purification method according to the invention, and relating to the prepared/implemented fusion protein.

In particular, an enzymatic cleavage site is inserted between the protein of interest and the protein tag.

A nucleic acid molecule encoding such a fusion protein, particularly suitable for implementation in the step of preparing the fusion protein of a purification method according to the invention, may in particular be obtained by any genetic engineering method conventional in itself.

Such a nucleic acid molecule may for example comprise, in the reading frame, a sequence chosen from the sequences SEQ ID No: 2 and SEQ ID No: 44, corresponding to the sequences encoding, respectively, the protein Mmi1 of Schizosaccharomyces pombe, of amino acid sequence SEQ ID No: 1, and the YTH+ domain of the latter, of amino acid sequence SEQ ID No: 9.

An expression vector comprising such a nucleic acid molecule, particularly suitable for implementation in the step of preparing the fusion protein of a purification method according to the invention, may be of any type known in itself for implementation in genetic engineering, in particular a plasmid, a cosmid, a virus, a bacteriophage, containing the elements necessary for the transcription and the translation of the sequence encoding the fusion protein according to the invention.

It notably comprises the following elements, functionally bound: a promoter located at 5′ of a nucleotide sequence encoding the fusion protein according to the invention, and termination signals of the transcription at 3′ of this sequence.

A host cell comprising such a fusion protein, such a nucleic acid molecule and/or such an expression vector, particularly suitable for implementation in the step of preparing the fusion protein of a purification method according to the invention, may also be a prokaryotic, particularly bacterial, cell in particular for the mass production of the fusion protein according to the invention, than a eukaryotic cell, this eukaryote possibly being lower or higher, for example a yeast, invertebrate or mammalian cell. In particular, cell lines expressing, in a stable, inducible or constitutive, or transiently, manner a fusion protein according to the invention fall within the scope of the invention.

The fusion protein implemented according to the invention may be prepared by any conventional method known to those skilled in the art. It may in particular be obtained by genetic engineering or by chemical synthesis.

A method for preparing such a fusion protein, which can be implemented to carry out the step of preparing the fusion protein of the purification method according to the invention, comprises the transfection of a host cell with a nucleic acid molecule as defined above or the transformation of a host cell with an expression vector as defined above; and the culture of this host cell under conditions allowing the expression of the targeted fusion protein.

Another object of the invention is a ribonucleic acid molecule containing at least one motif of UNAAAC nucleotide sequence, coupled to a capture ligand, for example to a biotin molecule.

This ribonucleic acid molecule may have any characteristic or combination of characteristics described above with reference to the purification method according to the invention, and relating to the implemented ribonucleic acid molecule.

Another aspect of the invention is a solid support for implementing a method for purifying a protein of interest according to the invention. A ribonucleic acid molecule containing at least one motif of UNAAAC nucleotide sequence is grafted onto this solid support.

This solid support may have any characteristic or combination of characteristics described above with reference to the purification method according to the invention, and relating to the implemented grafted solid support.

According to another aspect, the invention concerns a kit for the implementation of a method for purifying a protein of interest according to the invention. This kit contains a ribonucleic acid molecule containing at least one motif of UNAAAC nucleotide sequence grafted onto a solid support or coupled to a capture ligand, and at least one of the following constituents, for example these two constituents:
an expression vector comprising, under the control of a promoter, a nucleic acid molecule encoding a protein tag comprising at least, or consisting of: the protein Mmi1 of a microorganism of the genus Schizosaccharomyces, a fragment of this protein Mmi1 comprising at least the 173 C-terminal amino acids, or a protein of amino acid sequence having at least 90% identity with the amino acid sequence of said protein Mmi1 or of said fragment and capable of binding to a ribonucleic acid motif of UNAAAC nucleotide sequence; and a site allowing the insertion, at 5′ or at 3′ relative to said nucleic acid molecule encoding said protein tag, of a nucleic acid molecule encoding said protein of interest so as to allow the expression of a fusion protein containing said protein of interest and said protein tag;
and/or instructions for implementing the steps of a method according to the invention.

This kit may also contain, in the case in which the ribonucleic acid molecule is coupled to a capture ligand, a solid support on which an affinity partner of this capture ligand is grafted.

Each of these constituents may meet one or more of the characteristics described above with reference to the purification method according to the invention, and relating to this constituent.

The expression vector, allowing the cloning of the nucleic acid molecule encoding the protein of interest so as to form a chimeric nucleic acid molecule encoding the fusion protein, and the expression of the latter, may be of any type known in itself for implementation in genetic engineering, in particular a plasmid, a cosmid, a virus, a bacteriophage, etc. It contains the elements necessary for the cloning in a suitable insertion site of a nucleic acid molecule encoding the protein of interest, as well as the transcription and the translation of the chimeric sequence encoding this fusion protein “protein of interest-protein tag” (or “protein tag-protein of interest”) thus obtained. It comprises in particular the following elements, functionally bound: a promoter located at 5′ of a nucleic acid molecule encoding the protein tag according to the invention, an insertion site of a nucleic acid molecule acid encoding the protein of interest in the same reading frame as the nucleic acid molecule encoding the protein tag according to the invention, and termination signals of the transcription at 3′ of these elements.

The kit may also contain a host cell capable of being transformed by an expression vector comprising, under the control of a promoter: a nucleic acid molecule encoding a protein tag comprising at least, or consisting of, the protein Mmi1 of a microorganism of the genus Schizosaccharomyces, a fragment of this protein Mmi1 comprising at least the 173 C-terminal amino acids, or a protein of amino acid sequence having at least 90% identity with the amino acid sequence of said protein Mmi1 or of said fragment and capable of binding to a ribonucleic acid motif of UNAAAC nucleotide sequence; and a site allowing the insertion, at 5′ or at 3′ relative to this nucleic acid molecule encoding this protein tag, of a nucleic acid molecule encoding the protein of interest, this site being configured to allow the expression of a fusion protein containing the protein of interest and the protein tag.

This host cell may also be a prokaryotic, particularly bacterial, cell, in particular for the mass production of the fusion protein according to the invention, than a eukaryotic cell, this eukaryote possibly being lower or higher, for example a yeast, invertebrate or mammalian cell.

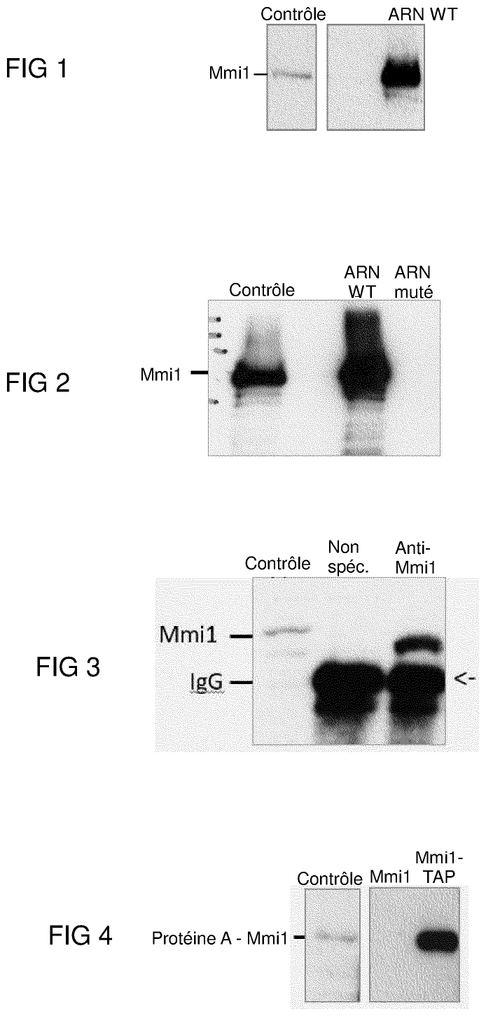

The characteristics and advantages of the invention will appear more clearly in the light of the examples of implementation below, provided simply by way of illustration and in no way limiting the invention, with the support of FIGS. 1 to 6, in which:

FIG. 1 shows a photograph of a western blot membrane, the revelation having been carried out by means of an anti-Mmi1 antibody, obtained after expression of the protein Mmi1 of S. pombe in E. coli, cell lysis, bringing into contact (right lane, “RNA WT”) or not (left lane, “Control”) for 15 min of the lysate diluted 1/100th with an RNA molecule “RNA WT” containing an UNAAAC motif (in accordance with the invention), recovery of the proteins bound to the RNA molecule, and separation of the proteins by SDS electrophoresis.

FIG. 2 shows a photograph of a western blot membrane, the revelation having been carried out by means of an anti-Mmi1 antibody, obtained after expression of the protein Mmi1 of S. pombe in E. coli, cell lysis, bringing into contact of the lysate diluted 1/10th with a RNA molecule “RNA WT” containing an UNAAAC motif (in accordance with the invention, middle lane) or a RNA molecule “mutated RNA” containing a CNAAAC motif (not in accordance with the invention—negative control, right lane) or absence of such bringing into contact (left lane, “Control”), recovery of the proteins bound to the RNA molecule, and separation of the proteins by SDS-PAGE electrophoresis.

FIG. 3 shows a photograph of a western blot membrane, the revelation having been carried out by means of an anti-Mmi1 antibody, obtained after expression of the protein Mmi1 of S. pombe in S. pombe, cell lysis, bringing into contact (or absence of bringing into contact: left lane, “Control”) for 1 hour of the lysate diluted 1/10th with an anti-Mmi1 antibody (right lane) or an antibody not directed against Mmi1 (human IgG, middle lane, “Not spec.”), recovery of proteins bound to the antibody, and separation of the proteins by SDS-PAGE electrophoresis.

FIG. 4 shows a photograph of a western blot membrane, the revelation having been carried out by means of an anti-Mmi1 antibody, obtained after expression of the protein Mmi1 of S. pombe in fusion with the protein A (right lane, “Mmi1-TAP”) or alone (middle lane, “Mmi1”) in S. pombe, cell lysis, bringing into contact (or absence of bringing into contact: left lane, “Control”) for 1 h of the lysate diluted 1/10th with IgG grafted on beads, recovery of the proteins bound to the IgG, and separation of the proteins by SDS-PAGE electrophoresis.

FIG. 5 shows photographs of western blot membranes, the revelation having been carried out by means of an anti-GFP antibody, obtained after expression in HEK293 cells, respectively, in a/ of the GFP and in b/ of the protein Mmi1 fused to the GFP.

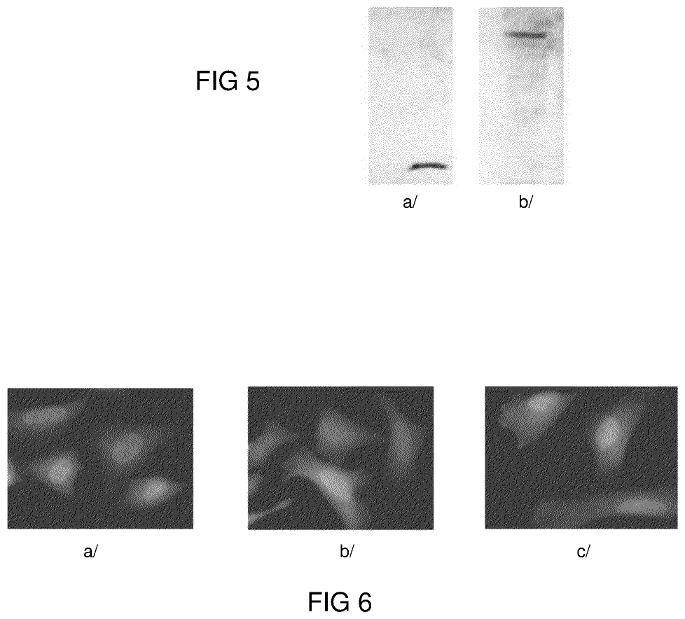

FIG. 6 shows images, acquired by fluorescence microscopy, of HEK293 cells overexpressing, in a/ the fusion protein of the GFP and of the protein Mmi1, the GFP being on the C-terminal side, in b/ the GFP alone, and in c/ the fusion protein of the GFP and of the protein Mmi1, the GFP being on the N-terminal side.

### A/ MATERIAL AND METHODS

### A.1/ Cells and Plasmids Used for the Production of the Proteins

Bacteria: Escherichia coli strain BL21 and plasmid pETM11 (the DNA sequences encoding the protein Mmi1 were cloned into this plasmid using the NcoI and EcoRI restriction sites);
Yeasts: Schizosaccharomyces Pombe;
Human cells: HeLa and HEK293 lines, and pEGFP-C3 and pEGFP-N3 plasmids (Clontech) (the DNA sequences encoding the protein Mmi1 were cloned into these plasmids using the XhoI and BamHI restriction sites).

In all experiments, the protein Mmi1 is that of S. pombe (complete protein Mmi1, of amino acid sequence SEQ ID No: 1).

### A.2/ Bacterial Expression

D1: transform the cells of the BL21 strain with the pETM11 plasmid containing Mmi1 following the standard procedure used for Top10 cells (by applying the conventional bacterial transformation protocol); D2: take a colony and inoculate it in 50 ml of LB medium supplemented with 50 mM kanamycin and let the culture grow at 37° C. with stirring (180 rpm); D3: transfer the inoculum into 200 ml of LB medium supplemented with 50 mM kanamycin and stir it again at 37° C. for 1 hour; check the optical density of the cells—when it reaches 0.8, take an aliquot of uninduced cells (5 ml), in order to constitute the negative control of induction (non-induced cells)—lower the temperature of the incubator to 18° C. and leave the culture stirring for 15 min before inducing the expression by adding IPTG—add 0.2 ml of 1M IPTG in a 200 ml culture (final concentration 1 mM) and leave the culture under stirring overnight; D4: centrifuge the cells in fractions of 50 ml at 5000 g for 20 min at 4° C., then freeze the pellets at −80° C.

A.3/ Binding of Mmi1 to RNA in E. coli

Cell lysis: a pellet of E. coli BL21 cells expressing the protein Mmi1 is thawed on ice. The cells are then lysed by sonication in 5 ml of lysis buffer (50 mM Tris pH 7.6, 150 mM KCl, 5 mM MgCl2, 1 mM EDTA, 1% Triton X-100, 10% glycerol, 5 mM DTT, 1 mM PMSF, 1 μg/ml of LABP (Protease inhibitor cocktail (Sigma): Aprotinin ref. 10236624001, Bestatine ref. 10874515001, Leupeptin ref. 10874515001, Pepstatin ref. 11359053001). The lysate is then transferred to a new tube of 15 ml and centrifuged for 10 min at 14000 g at 4° C., then alternately sonicated “10 s sonication/10 s rest” at 85% of the maximum power (sonicator Vibracell 75186, ThermoFisher) for a total sonication time of 2 min.

Preparation of biotinylated RNAs: for each immunoprecipitation, 100 ng of the RNAs named “RNA WT” and “mutated RNA” are denatured for 2 min at 90° C., then the denatured RNAs are incubated for 20 min at room temperature in the structuring buffer (10 mM Tris pH7, 0.1 M KCl, 10 mM MgCl₂) before being incubated with the lysate. The used RNAs were obtained from Eurogentec and have the following sequences:

RNA WT: 5′ Biot-GGAUCCUUAAACAGAUCU 3′ (SEQ ID No: 45) (artificial sequence, substrate for binding of Mmi1)—having an UNAAAC motif in accordance with the invention,
mutated RNA: 5′ Biot-GGAUCCCUAAACAGAUCU 3′ (SEQ ID No: 46) (artificial sequence, negative control for binding of Mmi1)—negative control not having an UNAAAC motif but a CNAAAC motif.

Binding to the RNA: a 1/100th or 1/10th dilution of the cell extract obtained at the end of the cell lysis step is carried out. The biotinylated RNA (100 ng) is added to 100 μl of cell extract, then the extract is incubated with stirring for 15 to 20 min at 4° C. The immunoprecipitation is carried out by adding 15 μl of 10 mg/ml Dynabeads® M-280 Streptavidin (Invitrogen) and incubating for 30 min with stirring. The beads are then washed three times with 1 ml of lysis buffer with 5 min of stirring for each wash. The protein Mmi1 associated with the RNA is eluted by boiling for 5 min in the 2× Laemmli SDS buffer. The efficiency of binding the protein to the RNA is analyzed by immunoblot (western blot), separation by SDS-PAGE electrophoresis being performed on 10% polyacrylamide gel for 1 h at 180 V. After transfer to nitrocellulose membrane, detection of the protein is carried out by means of an anti-Mmi1 antibody, as described in the publication by Touat-Todeschini et al, EMBO J, 2017, 36:2626-2641. For this purpose, the nitrocellulose membrane is incubated for one hour at room temperature with this anti-Mmi1 primary antibody diluted 1/1000 in TBS 0.1% tween (TBS-T) containing 10% FCS, followed by three washes of 5 min each with TBS-T. The membrane is then incubated for 45 min with a secondary antibody coupled to the HRP (DAKO, ref. P0448) diluted 1/5000 in TBS-T containing 1% milk, then washed again 3 times for 5 min with TBS-T.

### A.4/ Immunoprecipitation Experiments in Yeast

S. pombe cells in which Mmi1 was overexpressed by means of an inducible promoter (nmt1 promoter) inserted at the endogenous Mmi1 locus are used. Cells overexpressing Mmi1 in fusion with the TAP tag (for “Tandem Affinity Purification”, tandem affinity purification, composed of the protein A and of the CBP (calmodulin-binding protein), are also used, in which the TAP tag has been added to the endogenous sequence of the mmi1 gene by the conventional approach of homologous recombination of polymerization products by reaction in chain in yeast. These cells are cultured at an optical density (OD) of 1.2 in a total volume of 50 ml of YEA culture medium.

The following steps are all carried out at 4° C.

The lysis buffer (LysBuff) (100 mM HEPES pH 7.5, 20 mM MgCl₂, 10% Glycerol, 10 mM EGTA, 0.1 M EDTA, 0.4% NP-40, 150 mM NaCl, 1 mM DTT, 1 mM PMSF, 1 μg/ml LABP) is used for the lysis and for the washes.

Lysis of the cells: take the pellets in 400 μl of lysis buffer LysBuff (2×) (mix the tube gently if necessary), then add glass beads and stir for 2×30 s in a bead stirrer, with a rest time of 2 min in the ice between the two cycles. Pierce the bottom of the tubes with a 0.5 mm syringe and place the tubes of lysate into 5 ml round bottom tubes (chilled in ice). Centrifuge at 4° C. for 1 min at 3000 rpm, then transfer the lysates into new Eppendorf tubes. Centrifuge for 10 min at 13000 rpm at 4° C. The concentration of proteins in each obtained sample is estimated by the Bradford method, so as to use the same amount of total proteins in each immunoprecipitation experiment.

TAP immunoprecipitation: use 15 μl of IgG antibodies grafted onto Sepharose® resin beads (IgG Sepharose, Ref. 17-0969-01, GE-Healthcare) per immunoprecipitation experiment. Carry out 3 washes of the beads with 500 μl of 2×LysBuff lysis buffer. Mix the samples of proteins (the same amount of proteins in each sample) with 15 μl of beads prepared in the previous step. Stir gently at 4° C. for 1 hour.

Immunoprecipitation by anti-Mmi1 antibody: the proteins (the same quantity in each sample) are mixed with 2 μl of the aforementioned anti-Mmi1 antibody and the mixture is gently stirred for 1 h at 4° C. 20 μl of Sepharose® resin beads onto which the protein A is grafted (Sepharose® Protein A, Ref. 17-5280-01, GE-Healthcare) are added to the mixture, which is then stirred again for 1 h at 4° C.

Washes and elution: wash the beads 3 times with 500 μl of the 2×LysBuff, proceeding in the same way: washing for 5 min with gentle stirring then centrifugation for 2 min at 500 g. The elution is done by boiling for 5 min in 2× Laemmli SDS buffer. For detection by Western Blot, the same method as that described above with reference to the experiment of binding of Mmi1 to the RNA in E. coli. Detection of the TAP is done with an anti-TAP antibody.

### A.5/ Transfection of the Human Cells

The transfection is carried out according to the supplier's standard protocol, using the Lipofectamine® 3000 (ThermoFisher).

The cells are then either lysed for detection by the conventional Western blot method using an anti-GFP antibody (Roche #11814460001), or visualized directly on a slide under a fluorescence microscope, according to conventional protocols.

Visualization by Western blot and fluorescence microscopy is carried out after 48 h of transfection, according to conventional operating protocols.

### B/ EXPERIMENT 1

This experiment is carried out with the protein Mmi1 produced in E. coli.

The gene of sequence SEQ ID No: 2 is cloned into the plasmid, to allow the expression by the cells of the protein Mmi1 of S. pombe, of amino acid sequence SEQ ID No: 1 (complete protein Mmi1).

Schematically, an extract obtained by cell lysis of cells expressing the protein Mmi1 is bringing into contact with one or other of the biotinylated RNA molecules at 5′ “RNA WT” (in accordance with the invention) and “mutated RNA” (not in accordance with the invention, negative control), for a period of 15 min, then the RNA molecules (and the proteins attached to these molecules) are isolated from the medium by means of Dynabead® magnetic beads onto which the streptavidin is grafted, using the latter's strong ability to bind to the biotin. The proteins attached to the beads are eluted, and analyzed by western blot, after separation of the proteins by SDS-PAGE gel electrophoresis, by means of the anti-Mmi1 antibody.

The obtained results are shown on FIG. 1 for a 1/100th dilution of the cell lysis extract, for the RNA according to the invention “RNA WT”. The presence of the protein Mmi1 is clearly observed on the western blot membrane, and no background, demonstrating the binding specificity of the UNAAAC RNA motif with the protein Mmi1.

The obtained results are shown on FIG. 2 for a 1/10th dilution of the cell lysis extract, for the RNA according to the invention “RNA WT” and for the RNA not according to the invention “Mutated RNA”. The revelation by the anti-Mmi1 antibody reveals a band of high intensity corresponding to the molecular weight of the protein Mmi1 (54 kDa) when the RNA has an UNAAAC motif according to the invention (“RNA WT”). No band is observed at the molecular weight of the protein Mmi1 when the RNA molecule “mutated RNA” was implemented, which testifies to the binding specificity of the protein Mmi1 for the UNAAAC motif.

This experiment clearly demonstrates that the protein Mmi1 may be purified with a high degree of purity from a complex cell medium by interaction with a RNA molecule comprising an UNAAAC motif grafted onto a solid support.

### B/ EXPERIMENT 2—COMPARISON WITH ANTIBODY IMMUNOPRECIPITATION

This experiment is carried out with the endogenous protein Mmi1 of S. pombe, of amino acid sequence SEQ ID No: 1 (complete protein Mmi1).

Schematically, an extract obtained by cell lysis of cells expressing the protein Mmi1 is bringing into contact for 1 h with an anti-Mmi1 antibody.

After separation of the medium by means of Sepharose® beads onto which the protein A is grafted, the proteins attached to the beads are eluted and analyzed by western blot, after separation of the proteins by SDS-PAGE gel electrophoresis, by means of the anti-Mmi1 antibody.

The obtained results are shown on FIG. 3. We clearly observe, on the lane associated with the proteins captured by immunoprecipitation with the anti-Mmi1 antibody (“anti-Mmi1” lane), a band corresponding to the molecular weight of the protein Mmi1, which is not found on the other lanes. However, we also observe a band of very high intensity corresponding to a contamination by the IgG present in the initial cell extract (band indicated by an arrow on the right on the figure).

In comparison with this purification by the anti-Mmi1 antibody, the method according to the invention as implemented in experiment 1, the result obtained from which is illustrated on FIG. 1, allows, four times faster (15 min versus 1 h), to purify the protein Mmi1 with a much higher degree of purity.

### C/ EXPERIMENT 3—COMPARISON WITH A TAP IMMUNOPRECIPITATION

This experiment is carried out with the endogenous protein Mmi1 of S. pombe fused to the TAP.

Schematically, an extract obtained by lysis of cells expressing the protein Mmi1 in fusion with the protein A is bringing into contact for 1 hour with Sepharose® beads on which IgG are grafted, in order to isolate the protein Mmi1 from the medium by pulling benefit from the high affinity of the IgG for the protein A (Kd>10⁻⁹ M).

After separation of the beads from the medium, the proteins attached to the beads are eluted, and analyzed by western blot, after separation of the proteins by SDS-PAGE gel electrophoresis, by means of the anti-Mmi1 antibody.

The obtained results are shown on FIG. 4. We clearly observe, on the lane associated with the proteins captured by immunoprecipitation with the IgG (“Mmi1-TAP” lane), a band corresponding to the molecular weight of the fusion protein Protein A—Mmi1.

In comparison to this purification by the “Protein A—IgG” system, the method according to the invention as implemented in the experiment 1, the result obtained from which is illustrated on FIG. 1, allows, four times faster (15 min versus 1 h), to purify the protein Mmi1 with an equivalent degree of purity, and even slightly higher, in particular because it avoids any contamination with the IgG.

### D/ EXPERIMENT 4—FUSION WITH THE PROTEIN GFP

This experiment is carried out with the protein Mmi1 produced in the HEK293 cells, in fusion with the protein GFP.

For this purpose, the gene of sequence SEQ ID No: 2 is cloned into the plasmid pEGFP-C3 or pEGFP-N3, to allow the expression by HEK293 cells of the (complete) protein Mmi1 in fusion, at the C-terminal or at the N-terminal, with the protein GFP. A control GFP alone is also carried out.

The obtained cells are analyzed by western blot after cell lysis or observed by fluorescence microscopy to verify therein the localization of the GFP.

The obtained results are shown on FIG. 5 for the analysis by western blot after cell lysis and on FIG. 6 for the fluorescence analysis of the non-lysed cells.

These results demonstrate that the fusion protein of the GFP with Mmi1 does not have toxicity for human cells, that it is well expressed and localized in the cells. The results of the analysis by western blot confirm that the sizes of the detected proteins correspond to those expected (30 KDa for the GFP alone and 84 KDa for the fusion protein Mmi1-GFP), demonstrating the feasibility of the method according to the invention in mammalian cells.

1. A method for purifying a protein of interest successively comprising:
the preparation of a fusion protein comprising the protein of interest fused to a protein tag, the protein tag comprising at least: the protein Mmi1 of a microorganism of the genus Schizosaccharomyces, a fragment of the protein Mmi1 comprising at least the 173 C-terminal amino acids, or a protein of amino acid sequence having at least 90% identity with the amino acid sequence of the protein Mmi1 or of the fragment and capable of binding to a ribonucleic acid motif of UNAAAC nucleotide sequence,
the bringing into contact of the fusion protein with a ribonucleic acid molecule containing at least one motif of UNAAAC nucleotide sequence, so as to allow the affinity binding of the protein tag with the ribonucleic acid molecule, the ribonucleic acid molecule being grafted onto a solid support or coupled to a capture ligand,
where appropriate, the bringing into contact of the ribonucleic acid molecule coupled to a capture ligand with an affinity partner of the capture ligand grafted onto a solid support,
and the separation of the protein of interest and the solid support.

2. The purification method according to claim 1,
according to which an enzymatic cleavage site is inserted between the protein of interest and the protein tag.

3. The purification method according to claim 2, according to which the separation of the protein of interest and the solid support is carried out by cleavage at the enzymatic cleavage site.

4. The purification method according to claim 1, according to which a cleavage site is inserted between the ribonucleic acid molecule containing at least one motif of UNAAAC nucleotide sequence and the solid support or the ligand of interest, and the separation of the protein of interest and the solid support is carried out by cleavage at the cleavage site.

5. The purification method according to claim 1, according to which the solid support is a chromatography support.

6. The purification method according to claim 1, according to which the ribonucleic acid molecule contains one or more repeats of the motif of UNAAAC sequence.

7. The purification method according to claim 1, according to which the ribonucleic acid molecule comprises at least one chemically modified nucleotide and/or at least one locked nucleic acid and/or at least one phosphorothiate internucleotide bond.

8. The purification method according to claim 1, according to which the protein Mmi1 comes from a species chosen from Schizosaccharomyces pombe, Schizosaccharomyces japonicus, Schizosaccharomyces octosporus and Schizosaccharomyces cryophilus.

9. The method according to claim 1, according to which the fragment of the protein Mmi1 comprising at least the 173 C-terminal amino acids has an amino acid sequence comprising, or consisting of, the amino acid sequence SEQ ID No: 9, SEQ ID No: 10, SEQ ID No: 11 or SEQ ID No: 12.

10. The method according to claim 1, according to which the protein tag contains at least one domain chosen from the domains of amino acid sequence:
RSVWXaa₁Xaa₂Xaa₃Xaa₄Xaa₅Xaa₆P (SEQ ID No: 13), where Xaa₁ represents a threonine, serine or alanine residue, Xaa₂ represents a threonine, arginine, lysine or serine residue, Xaa₃ represents a histidine or arginine residue, Xaa₄ represents a threonine or proline residue, Xaa₅ represents a glycine, alanine or arginine residue and Xaa₆ represents a glutamic acid or aspartic acid residue,
FXaa₇SPLKRXaa₈APXaa₉SXaa₁₀Xaa₁₁Xaa₁₂Xaa₁₃Xaa₁₄Xaa₁₅R (SEQ ID No: 14), where Xaa₇ represents a serine or threonine residue, Xaa₈ represents a proline or glycine residue, Xaa₉ represents a glutamic acid or aspartic acid residue, Xaa₁₀ represents a histidine, arginine or lysine residue, Xaa₁₁ represents an aspartic acid or glutamic acid residue, Xaa₁₂ represents an alanine or tyrosine residue, Xaa₁₃ is zero or represents a proline residue, Xaa₁₄ represents an isoleucine or methionine residue and Xaa₁₅ represents a glycine or aspartic acid residue,
YDFXaa₁₆RHCTDYGHSYXaa₁₇WPYFRSXaa₁₈RREXaa₁₉Xaa₂₀Xaa₂₁Y (SEQ ID No: 15), where Xaa₁₆ represents a serine, threonine or tyrosine residue, Xaa₁₇ represents a glutamic acid or aspartic acid residue, Xaa₁₈ represents a leucine or valine residue, Xaa₁₉ is zero or represents a serine residue, Xaa₂₀ represents a leucine or methionine residue and Xaa₂₁ represents an arginine, leucine or methionine residue,
QPPXaa₂₂KRRTLXaa₂₃Xaa₂₄P (SEQ ID No: 16), where Xaa₂₂ represents a proline, serine or leucine residue, Xaa₂₃ represents a serine or leucine residue and Xaa₂₄ represents a proline or serine residue,
Xaa₂₅AXaa₂₆Xaa₂₇SPXaa₂₈Xaa₂₉Xaa₃₀Xaa₃₁PXaa₃₂Xaa₃₃H (SEQ ID No: 17), where Xaa₂₅ represents an arginine or aspartic acid residue, Xaa₂₆ represents a serine or glycine residue, Xaa₂₇ represents a histidine or aspartic acid residue, Xaa₂₈ represents a serine, glycine or leucine residue, Xaa₂₉ represents a leucine or phenylalanine residue, Xaa₃₀ represents a leucine, isoleucine or serine residue, Xaa₃₁ represents a glutamic acid or aspartic acid residue, Xaa₃₂ represents a tyrosine or threonine residue and Xaa₃₃ represents an alanine or threonine residue,
RXaa₃₄EKPKXaa₃₅RAXaa₃₆TPPP (SEQ ID No: 18), where Xaa₃₄ represents a lysine or arginine residue, Xaa₃₅ represents an alanine, proline or threonine residue and Xaa₃₆ represents a serine or proline residue.

11. A solid support for implementing a method for purifying a protein of interest according to claim 1, wherein a ribonucleic acid molecule containing at least one motif of UNAAAC nucleotide sequence is grafted onto the solid support.

12. A kit for implementing a method for purifying a protein of interest according to claim 1, wherein it contains a ribonucleic acid molecule containing at least one motif of UNAAAC nucleotide sequence grafted onto a solid support or coupled to a capture ligand, and at least one of the following constituents:
an expression vector comprising, under the control of a promoter, a nucleic acid molecule encoding a protein tag comprising at least: the protein Mmi1 of a microorganism of the genus Schizosaccharomyces, a fragment of the protein Mmi1 comprising at least the 173 C-terminal amino acids, or a protein of amino acid sequence having at least 90% identity with the amino acid sequence of the protein Mmi1 or of the fragment and capable of binding to a ribonucleic acid motif of UNAAAC nucleotide sequence; and a site allowing the insertion, at 5′ or at 3′ relative to the nucleic acid molecule encoding the protein tag, of a nucleic acid molecule encoding the protein of interest so as to allow the expression of a fusion protein containing the protein of interest and the protein tag;
instructions for implementing the steps of the method.

13. The kit according to claim 12, further containing a host cell capable of being transformed by an expression vector comprising, under the control of a promoter, a nucleic acid molecule encoding a protein tag comprising at least: the protein Mmi1 of a microorganism of the genus Schizosaccharomyces, a fragment of the protein Mmi1 comprising at least the 173 C-terminal amino acids, or a protein of amino acid sequence having at least 90% identity with the amino acid sequence of the protein Mmi1 or of the fragment and capable of binding to a ribonucleic acid motif of UNAAAC nucleotide sequence; and a site allowing the insertion, at 5′ or at 3′ relative to the nucleic acid molecule encoding the protein tag, of a nucleic acid molecule encoding the protein of interest so as to allow the expression of a fusion protein containing the protein of interest and the protein tag.
